(19) United States
(12) Patent Application Publication
(10) Pub. No.: US 2020/0138802 A1
(43) Pub. Date: May 7, 2020

(54) Aggressive Lipid Lowering Therapy in Coronary Artery Disease

(71) Applicant: Kowa Company, Ltd., Aichi (JP)

(72) Inventors: Yasushi Saito, Chiba (JP); Ryozo Nagai, Tokyo (JP); Masunori Matsuzaki, Yamaguchi (JP); Takeshi Kimura, Kyoto (JP); Hiroaki Shimokawa, Miyagi (JP); Hiroyuki Daida, Tokyo (JP); Satoshi Iimuro, Tokyo (JP); Hiroshi Iwata, Tokyo (JP); Yukio Ozaki, Aichi (JP); Ichiro Sakuma, Hokkaido (JP); Yoshihisa Nakagawa, Nara (JP); Kiyoshi Hibi, Kanagawa (JP); Takafumi Hiro, Yamaguchi (JP); Yoshihiro Fukumoto, Fukuoka (JP); Seiji Hokimoto, Kumamoto (JP); Katsumi Miyauchi, Tokyo (JP); Yasuo Ohashi, Tokyo (JP)

(21) Appl. No.: 16/672,469

(22) Filed: Nov. 3, 2019

Related U.S. Application Data
(60) Provisional application No. 62/756,739, filed on Nov. 7, 2018.

(51) Int. Cl. A61K 31/47; A61P 3/06

(52) U.S. Cl. CPC A61K 31/47; A61P 3/06

(57) ABSTRACT

Methods of further reducing cardiovascular risk in subjects with coronary artery disease on moderate intensity statins.

### FIELD OF THE INVENTION

The present invention relates to methods of further reducing cardiovascular risk in patients with coronary artery disease on moderate intensity statins.

### BACKGROUND OF THE INVENTION

Cardiovascular (“CV”) death is an important medical and social problem and is increasing every year, including in Japan. Although widespread use of new and effective drug treatments and percutaneous coronary intervention (“PCI”) have improved outcomes in patients with coronary artery disease (“CAD”), the recurrence rate in patients for secondary prevention remains high compared with patients for primary prevention. (1-3)

Many epidemiological studies have proven that coronary deaths and CV events are associated with elevated levels of low-density lipoprotein cholesterol (LDL-C). Furthermore, large-scale clinical trials have found that lipid-lowering therapy using statins reduces CV events. (4-11) In the J-LIT and MUSASHI-AMI studies in Japan, the recurrence risk for CV events was significantly decreased when LDL-C levels were reduced to below 100 mg/dL (2.6 mmol/L) with statin treatment. (1,12) Based on these findings, the guidelines of the Japan Atherosclerosis Society state that LDL-C levels should be 100 mg/dL or lower for secondary prevention. (13) This target is higher than the targets specified in European and former American guidelines. (14,15)

Secondary prevention trials such as the A to Z, TNT, IDEAL, and PROVE-IT studies have compared the rate of CV events between high intensity and moderate intensity lipid-lowering treatments. (16-19) In a meta-analysis of these trials, high intensity lipid-lowering treatment significantly reduced the rate of CV events by 16% compared with moderate intensity lipid-lowering therapy. (20) CV events were correlated with LDL-C reduction, both in patients with acute coronary syndrome (ACS) and patients with chronic phase CAD. (21) Therefore, for secondary prevention, European guidelines recommend a target LDL-C of less than 70 mg/dL, and American College of Cardiology (ACC)/American Heart Association (AHA) guidelines recommend high-intensity statin therapy. (14,22) It has been reported that 4 mg of pitavastatin can reduce LDL-C by 42.9% from baseline. (29)

Because the CV event rate in Asian populations is much lower than that in European and American populations, (23,24) it is important to confirm the benefits of statin therapy in Asian populations. In Japan, no large-scale outcome studies on the use of statins for secondary prevention have been conducted. However, several trials have used intravascular ultrasound to show that intensive LDL-C reduction with statins leads to plaque regression. The ESTABLISH, JAPAN-ACS, and COSMOS trials demonstrated that lowering LDL-C to 70-80 mg/dL by aggressive lipid-lowering treatment with moderately high-dose statins significantly reduced coronary plaque volume. (25-27) Moreover, the extended ESTABLISH trial suggested that plaque regression during the randomized period correlated with reduced long-term CV events. (28) However, because no prospective clinical trials comparing high-dose and low-dose statin therapy have been conducted in Asia, it is unknown whether high-dose statin therapy can safely improve clinical outcomes in Asian populations.

It is an object of the present invention to reduce CV risk, further than CV risk is reduced by moderate intensity statin therapy.

Another object of the present invention is to identify patient populations in whom such risk reduction strategies are effective.

Another object of the present invention is to reduce CV risk, in subjects with established coronary artery disease and/or moderately elevated LDL-C levels.

Another object of the present invention is to identify unique clinical benefits of high intensity statin therapy, and to implement such therapy to provide such benefits.

Still further objects are to modulate biomarkers of cardiovascular risk, employing high intensity statin therapy, in high risk patients.

### SUMMARY OF INVENTION

Thus, in a first principal embodiment the invention provides a method of preventing an adverse cardiovascular event in a subject on a dosage of 1 mg/day pitavastatin or a pharmaceutically acceptable salt thereof, wherein the subject has an LDL-C concentration less than 120 mg/dL, comprising increasing the dosage to 4 mg/day for a therapeutically effective period of time. The subject preferably has coronary artery disease.

In a second principal embodiment the invention provides a method of preventing an adverse cardiovascular event in a subject with stable coronary artery disease, comprising administering to the subject 4 mg/day of pitavastatin or a pharmaceutically acceptable salt thereof for a therapeutically effective period of time. Prior to administering the 4 mg/day the subject is preferably on a dosage of 1 mg/day pitavastatin or a pharmaceutically acceptable salt thereof and has an LDL-C concentration less than 120 mg/dL.

In a third principal embodiment the invention provides a method of preventing an adverse cardiovascular event in a subject on a dosage of 1 mg/day pitavastatin or a pharmaceutically acceptable salt thereof, wherein the subject has stable coronary artery disease and an LDL-C concentration less than 120 mg/dL, comprising increasing the dosage to 4 mg/day for a therapeutically effective period of time.

In a fourth principal embodiment the invention provides a method of modulating a biomarker of cardiovascular risk in a subject with stable coronary artery disease, comprising administering to the subject 4 mg/day of pitavastatin or a pharmaceutically acceptable salt thereof for a therapeutically effective period of time, wherein the modulating is selected from (a) decreasing LDL-C levels in the subject; (b) increasing HDL-C levels in the subject; (c) decreasing TG levels in the subject; (d) decreasing hs-CRP levels in the subject; or (e) all or a combination thereof. Prior to administering the 4 mg/day the subject is preferably on a dosage of 1 mg/day pitavastatin or a pharmaceutically acceptable salt thereof and has an LDL-C concentration less than 120 mg/dL.

Additional advantages of the invention are set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying drawings, which are incorporated in and constitute a part of this specification, illustrate several embodiments of the invention and together with the description serve to explain the principles of the invention.

FIG. 1 is a logical flow-chart depicting the rationale and design of the human clinical trial described in Example 1.

FIG. 2 charts LDL-C levels starting at baseline in two separate groups of subjects, each initially stabilized on 1 mg pitavastatin per day, the first group remaining on 1 mg pitavastatin/day for the duration of the trial, the second increasing the dose to 4 mg pitavastatin/day for the duration of the trial, as described in Example 1.

FIG. 3 charts HDL-C levels starting at baseline in two separate groups of subjects, each initially stabilized on 1 mg pitavastatin per day, the first group remaining on 1 mg pitavastatin/day for the duration of the trial, the second increasing the dose to 4 mg pitavastatin/day for the duration of the trial, as described in Example 1.

FIG. 4 charts TG levels starting at baseline in two separate groups of subjects, each initially stabilized on 1 mg pitavastatin per day, the first group remaining on 1 mg pitavastatin/day for the duration of the trial, the second increasing the dose to 4 mg pitavastatin/day for the duration of the trial, as described in Example 1.

FIG. 5 charts hs-CRP levels starting at baseline in two separate groups of subjects, each initially stabilized on 1 mg pitavastatin per day, the first group remaining on 1 mg pitavastatin/day for the duration of the trial, the second increasing the dose to 4 mg pitavastatin/day for the duration of the trial, as described in Example 1.

FIG. 6 charts the cumulative incidence over time of CV events (CV death, myocardial infarction (“MI”), ischemic stroke, and unstable angina (“UA”) requiring emergency hospitalization) in two separate groups, each initially stabilized on 1 mg pitavastatin per day, the first group remaining on 1 mg pitavastatin/day for the duration of the trial, the second increasing the dose to 4 mg pitavastatin/day for the duration of the trial, as described in Example 1.

FIG. 7 charts the cumulative incidence over time of CV events (CV death, MI, ischemic stroke, UA requiring emergency hospitalization, and coronary revascularization) in two separate groups, each initially stabilized on 1 mg pitavastatin per day, the first group remaining on 1 mg pitavastatin/day for the duration of the trial, the second increasing the dose to 4 mg pitavastatin/day for the duration of the trial, as described in Example 1.

FIG. 8 charts the individual CV events in two separate groups, each initially stabilized on 1 mg pitavastatin per day, the first group remaining on 1 mg pitavastatin/day for the duration of the trial, the second increasing the dose to 4 mg pitavastatin/day for the duration of the trial, as described in Example 1.

FIG. 9 charts the subgroup analyses of primary endpoint (CV death, MI, ischemic stroke, UA requiring emergency hospitalization) in two separate groups, each initially stabilized on 1 mg pitavastatin per day, the first group remaining on 1 mg pitavastatin/day for the duration of the trial, the second increasing the dose to 4 mg pitavastatin/day for the duration of the trial, as described in Example 1.

### DETAILED DESCRIPTION

### Definitions and Use of Terms

As used in this specification and in the claims which follow, the singular forms “a,” “an” and “the” include plural referents unless the context clearly dictates otherwise.

As used in the specification and claims, the singular forms “a,” “an,” and “the” include plural references unless the context clearly dictates otherwise. For example, the term “a pharmaceutical excipient” refers to one or more pharmaceutical excipients for use in the presently disclosed formulations and methods.

As used in this specification and in the claims which follow, the word “comprise” and variations of the word, such as “comprising” and “comprises,” means “including but not limited to,” and is not intended to exclude, for example, other additives, components, integers or steps. When an element is described as comprising a plurality of components, steps or conditions, it will be understood that the element can also be described as comprising any combination of such plurality, or “consisting of” or “consisting essentially of” the plurality or combination of components, steps or conditions.

The use of numerical values in this application can be preceded by the word “about”. Also, the disclosure of ranges is intended as a continuous range including every value between the minimum and maximum values recited as well as any ranges that can be formed by such values. Also, disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

As used herein, “cardiovascular events” or “CV events” includes any adverse CV event including CV death; nonfatal myocardial infarction (“MI”); nonfatal ischemic stroke; unstable angina (“UA”) (e.g., UA determined to be caused by myocardial ischemia by, for example, invasive or non-invasive testing, and requiring hospitalization); cardiac arrest; peripheral CV disease requiring intervention, angioplasty, bypass surgery or aneurysm repair; and onset of new congestive heart failure.

As used herein, “preventing the occurrence of a CV event” includes reducing the risk of a CV event, delaying the incidence or occurrence of a CV event, and minimizing the severity of CV event. It also refers to a time interval beginning at (a) an initial administration of a therapeutically effective amount of pitavastatin or a pharmaceutically acceptable salt thereof as disclosed herein to the subject to (b) a cardiovascular event in the subject greater than or substantially greater than a control time interval beginning at (a′) initial administration of a placebo to control subjects to (b′) a cardiovascular event in the control subjects.

As used herein, “therapeutically effective amount” refers to an amount sufficient to elicit the desired biological response in a subject. The therapeutically effective amount or dose depends on the age, sex and weight of the subject, and the current medical condition of the subject. The skilled artisan can determine appropriate amount or dose depending on the above factors based on his or her knowledge and the teachings contained herein.

When a dose of pitavastatin or pharmaceutically acceptable salt thereof is stated in this document, it will be understood that the dose is based on the weight of the free base of pitavastatin. Thus, a 1 mg dose of pitavastatin or a pharmaceutically acceptable salt thereof contains 1 mg of pitavastatin as the free base, regardless of whether the pitavastatin is present as the free base or a salt. A 1 mg dose of pitavastatin calcium contains 1 mg of the free base of pitavastatin, and 1.045 mg of pitavastatin calcium. A preferred form of pitavastatin in all embodiments of the present invention is pitavastatin calcium.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use. “Pharmaceutically acceptable salts” means salts that are pharmaceutically acceptable, as defined above, and which possess the desired pharmacological activity.

The terms “treating” and “treatment,” when used herein, refer to the medical management of a subject with the intent to cure, ameliorate, stabilize, or prevent a disease, pathological condition, or disorder (collectively “disorder”). These terms include active treatment, that is, treatment directed specifically toward the improvement of a disorder, and also include causal treatment, that is, treatment directed toward removal of the cause of the associated disorder. In addition, this term includes palliative treatment, that is, treatment designed for the relief of symptoms rather than the curing of the disorder; preventative treatment, that is, treatment directed to minimizing or partially or completely inhibiting or delaying the development of the disorder; and supportive treatment, that is, treatment employed to supplement another specific therapy directed toward the improvement of the disorder. When a method is said to be “reducing” CV risk, it will be understood that the method also “treats CV disease.”

All analyte measurements recited herein, when used to define a subject described herein, are measured at the beginning of therapy according to the claim, i.e., when the pitavastatin dose is increased.

Unless stated herein to the contrary, all analyte measurements are taken in the fasting state, and are based on the concentration of the analyte in plasma or serum. The fasting state means that the subject has not eaten anything in from 8 to 12 hours, except for water. Standard methods of measuring analytes can be found in Lab Protocols for NHANES 2003-2004 data published by the United States Centers for Disease Control.

As used herein, the term “significantly” refers to a level of statistical significance. The level of statistical significant can be, for example, of at least p<0.05, of at least p<0.01, of at least p<0.005, or of at least p<0.001. Whenever a numeric value or endpoint is specified herein, it will be understood in a preferred embodiment to have a degree of statistical significance of at least p<0.05.

### Discussion of Principal Embodiments

The invention is described herein in terms of principal embodiments and subembodiments. It will be understood that each of the subembodiments can modify any of the principal embodiments, unless such modification is logically inconsistent or expressly disallowed in this document. It will be further understood that the principal embodiments can be combined in any manner, and that the subembodiments can be combined in any manner to further modify any of the principal embodiments, unless such combination is logically inconsistent or expressly disallowed in this document.

In a first principal embodiment the invention provides a method of preventing an adverse cardiovascular event in a subject on a dosage of 1 mg/day pitavastatin or a pharmaceutically acceptable salt thereof, wherein the subject has an LDL-C concentration less than 120 mg/dL, comprising increasing the dosage to 4 mg/day for a therapeutically effective period of time. The subject preferably has coronary artery disease.

In a second principal embodiment the invention provides a method of preventing an adverse cardiovascular event in a subject with stable coronary artery disease, comprising administering to the subject 4 mg/day of pitavastatin or a pharmaceutically acceptable salt thereof for a therapeutically effective period of time. Prior to administering the 4 mg/day the subject is preferably on a dosage of 1 mg/day pitavastatin or a pharmaceutically acceptable salt thereof and has an LDL-C concentration less than 120 mg/dL.

In a third principal embodiment the invention provides a method of preventing an adverse cardiovascular event in a subject on a dosage of 1 mg/day pitavastatin or a pharmaceutically acceptable salt thereof, wherein the subject has stable coronary artery disease and an LDL-C concentration less than 120 mg/dL, comprising increasing the dosage to 4 mg/day for a therapeutically effective period of time.

In a fourth principal embodiment the invention provides a method of modulating a biomarker of cardiovascular risk in a subject with stable coronary artery disease, comprising administering to the subject 4 mg/day of pitavastatin or a pharmaceutically acceptable salt thereof for a therapeutically effective period of time, wherein the modulating is selected from (a) decreasing LDL-C levels in the subject; (b) increasing HDL-C levels in the subject; (c) decreasing TG levels in the subject; (d) decreasing hs-CRP levels in the subject; or (e) all or a combination thereof. Prior to administering the 4 mg/day the subject is preferably on a dosage of 1 mg/day pitavastatin or a pharmaceutically acceptable salt thereof and has an LDL-C concentration less than 120 mg/dL.

### Discussion of Subembodiments

Each of the following subembodiments can apply to limit each and every one of the foregoing principal embodiments, although it will be understood that the subembodiment will not apply if it is already wholly subsumed by the principal embodiment. It will also be understood that the subembodiments can be combined with each other in any manner that is logically and mathematically possible, adopting the more restrictive value when two values for the same limitation are expressed, to define further subembodiments.

In any of the principal embodiments or subembodiments of the present invention, the subject preferably has stable coronary artery disease, preferably characterized by (a) previous ACS, such as acute myocardial infarction or unstable angina; (b) a previous coronary revascularization procedure (PCI or coronary artery bypass grafting (“CABG”)); or (c) atherosclerotic CAD detected by coronary artery angiography with at least 75% stenosis in major epicardial coronary arteries.

In one subembodiment, the methods of the present invention prevent CV death. In another subembodiment, the methods of the present invention prevent myocardial infarction. In another subembodiment, the methods of the present invention prevent unstable angina (UA) requiring emergency hospitalization. In another subembodiment, the methods of the present invention prevent any of CV death, myocardial infarction, or unstable angina (UA) requiring emergency hospitalization. In another subembodiment, the methods of the present invention prevent any of CV death, myocardial infarction, unstable angina (UA) requiring emergency hospitalization, or coronary revascularization. In another subembodiment, the methods of the present invention prevent death from any cause. In another subembodiment, the methods of the present invention prevent coronary revascularization (all). In another subembodiment, the methods of the present invention prevent coronary revascularization (non-TLR). In another subembodiment, the methods of the present invention prevent coronary revascularization (TLR).

The methods can also be practiced in people depending on their age. Thus, in one subembodiment the method is practiced in a subject who has an age less than 65. In another subembodiment the method is practiced in a subject who has an age greater than or equal to 65.

The methods can also be practiced in people depending on their type-II diabetes status. Thus, in one subembodiment the method is practiced in a subject who has type-II diabetes. In another subembodiment the method is practiced in a subject without type II diabetes.

The methods can also be practiced in people based on their LDL-C concentration. Thus, in one subembodiment the method is practiced in a subject who has an LDL-C concentration less than 95 mg/dL. In another subembodiment the method is practiced in a subject who has an LDL-C concentration greater than or equal to 95 mg/dL.

The methods can also be practiced in people based on their hs-CRP concentration. Thus, in one subembodiment the method is practiced in a subject who has an hs-CRP concentration less than 1 mg/L. In another subembodiment the method is practiced in a subject who has an LDL-C concentration greater than or equal to 1 mg/L.

The methods can also be practiced in people based on their HDL-C concentration. Thus, in one subembodiment the method is practiced in a subject who has an HDL-C concentration less than or equal to 40 mg/dL. In another subembodiment the method is practiced in a subject who has an HDL-C concentration greater than 40 mg/dL.

The methods can also be practiced in people based on their TG concentration. Thus, in one subembodiment the method is practiced in a subject who has an TG concentration less than 150 mg/dL. In another subembodiment the method is practiced in a subject who has an TG concentration greater than or equal to 150 mg/dL.

The methods can also be practiced in people based on their body mass index (“BMI”). Thus, in one subembodiment the method is practiced in a subject who has a BMI less than 25 kg/m². In another subembodiment the method is practiced in a subject who has a BMI greater than or equal to 25 kg/m². In another subembodiment the subject has a body mass index less than 28 or 26 kg/m².

In another subembodiment the subject has hypertension. In another subembodiment the subject does not have hypertension. In another subembodiment the subject has a history of acute coronary syndrome (ACS). In another subembodiment the subject does not have a history of acute coronary syndrome (ACS). In another subembodiment the subject has had a coronary revascularization. In another subembodiment the subject has not had a coronary revascularization. In another subembodiment the subject has had an ischemic stroke. In another subembodiment the subject has not had an ischemic stroke. In another subembodiment the subject has peripheral vascular disease. In another subembodiment the subject does not have peripheral vascular disease. In another subembodiment the subject has chronic kidney disease (CKD) (eGFR<60 mL/min/1.73 m²). In another subembodiment the subject does not have chronic kidney disease (CKD) (eGFR<60 mL/min/1.73 m²). In another subembodiment the subject is on aspirin therapy. In another subembodiment the subject is not on aspirin therapy. In another subembodiment the subject is on dual antiplatelet therapy (DAPT). In another subembodiment the subject is not on dual antiplatelet therapy (DAPT).

In other subembodiments the methods are practiced to modulate biomarker levels in an affected subject. Thus, in one subembodiment the methods further comprise (a) decreasing LDL-C levels in the subject; (b) increasing HDL-C levels in the subject; (c) decreasing TG levels in the subject; (d) decreasing hs-CRP levels in the subject; or (e) all or a combination thereof. LDL-C, HDL-C, TG, and hs-CRP levels in the subject prior to a 4 mg dose of pitavastatin are preferably as described elsewhere in this document (i.e. LDL-C<95 mg/dL or ≥95 mg/dL; hs-CRP<1 mg/L or ≥1 mg/L; HDL-C≤40 mg/dL or >40 mg/dL; and/or TG<150 mg/dL or ≥150 mg/dL).

In other subembodiments the methods are practiced in individuals based on their ethnic ancestry.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated, and are intended to be purely exemplary of the invention and are not intended to limit the scope of what the inventors regard as their invention.

### Example 1. Evaluation of Aggressive Lipid Lowering Therapy in Coronary Artery Disease in Japanese Patients

### Study Design and Objectives:

This study was a prospective, multicenter, randomized, open-label, blinded-endpoint, physician-initiated phase 4 trial designed to investigate whether high-dose statin therapy could reduce CV events in patients with stable CAD as compared with low-dose statin therapy. We hypothesized that aggressive lipid lowering treatment with pitavastatin 4 mg/day would reduce the incidence of the composite endpoint comprised of CV death, non-fatal myocardial infarction (MI), non-fatal ischemic stroke, and unstable angina requiring emergency hospitalization. The control group received pitavastatin 1 mg/day.

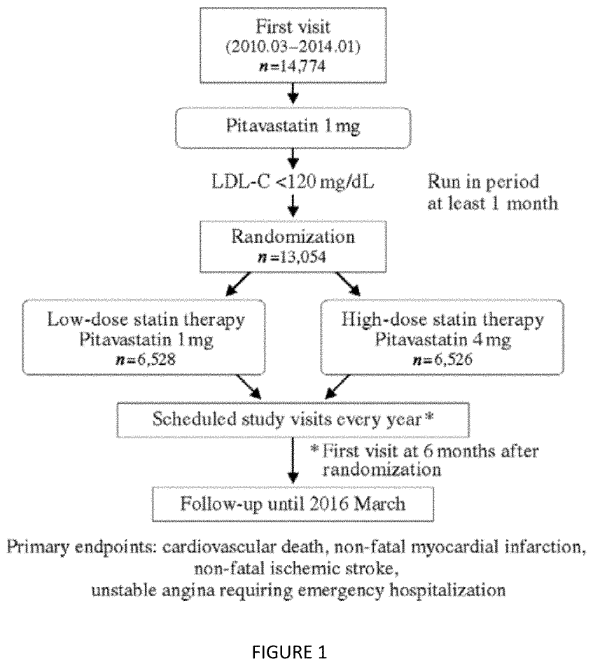

Ethical approval was obtained from Public Health Research Foundation ethics review committee and the relevant ethics committees at all participating sites. All patients provided written informed consent. The study was conducted in accordance with ethical principals in the Declaration of Helsinki. The overall design of the study is depicted in FIG. 1.

### Study Population and Patient Selection:

Men and women aged 20 to 80 years with clinically evident stable CAD and elevated LDL-C were eligible for this study. CAD was defined as 1) previous ACS, such as acute myocardial infarction or unstable angina; 2) a previous coronary revascularization procedure (PCI or coronary artery bypass grafting (“CABG”)); or 3) atherosclerotic CAD detected by coronary artery angiography with at least 75% stenosis in major epicardial coronary arteries (American Heart Association classification). (30) Patients with elevated LDL-C at entry were defined as (1) patients not receiving chronic lipid-lowering therapy with LDL-C≥140 mg/dL, (2) patients with LDL-C≥100 mg/dL and considered by the attending physician to need lipid-lowering therapy, or (3) patients being treated with lipid-lowering therapy.

Exclusion criteria included (1) any planned coronary revascularization procedure, (2) active malignancy, (3) contraindication for pitavastatin (hypersensitivity to pitavastatin, severe liver disease or hepatic dysfunction, concurrent cyclosporine use, or suspected or confirmed pregnancy or current breastfeeding), (4) severe congestive heart failure (ejection fraction<30% or NYHA class≥3), (5) current hemodialysis, (6) familial hypercholesterolemia, (7) current participation in another clinical trial, (8) current use of any prohibited drug (lipid-lowering drug other than pitavastatin) that could not be discontinued during the study, and (9) any other finding that would make the patient unsuitable for this study in the opinion of the investigators.

### Randomization and Treatment Protocol:

Participants were recruited at 733 hospitals in Japan. During screening visits, informed consent and baseline medical history were obtained, and a clinical examination and laboratory testing were performed. Blood samples were collected to determine fasting lipid levels and the patient's standard clinical profile. After discontinuation of all previous lipid-lowering therapies, all eligible patients started receiving treatment with pitavastatin 1 mg/day on an open-label basis for at least 1 month (run-in period). At the end of the run-in period (week 0), those patients with LDL-C levels below 120 mg/dL, measured by a central laboratory using Friedwald's formula, were eligible for randomization. Patients with prior ACS or coronary revascularization could be randomized beyond 3 months after the index event. Randomization was not allowed for patients with poor adherence (less than 50%) for the study drug during the run-in period or for patients with the primary endpoint events during the run-in period.

Patients were randomized in a 1:1 ratio to either pitavastatin 4 mg or pitavastatin 1 mg once daily. Randomized treatment assignment was stratified by 5 factors: 1) institution, 2) prior statin use, 3) age (<65 or ≥65), 4) diabetes, and 5) sex. Patients were to be followed for 3 years, and study visits were scheduled at 6 months for the first visit and every year thereafter. At each visit, information on vital signs, clinical endpoints, adverse events, and concurrent medication was collected. In addition, physical examinations and electrocardiograms were performed and blood specimens were collected at 6 months and every 12 months thereafter for lipid and other laboratory tests.

### Endpoints:

The primary endpoint of the trial was a composite of CV death, non-fatal MI, non-fatal ischemic stroke, or unstable angina requiring urgent hospitalization. The secondary endpoints included: (1) composite CV events (CV death, non-fatal MI, non-fatal ischemic stroke, unstable angina requiring urgent hospital admission, and coronary revascularization for non-target lesions at previous coronary revascularization), (2) composite coronary events (coronary death, non-fatal MI, unstable angina requiring urgent hospital admission, and coronary revascularization), (3) composite cerebrovascular events (fatal or non-fatal stroke and transient ischemic attack requiring hospital admission), (4) death (all cause death, CV death, cardiac death, death from coronary heart disease (“CHD”)), (5) individual cardiac events (fatal and non-fatal MI, unstable angina requiring urgent hospital admission, hospitalization with primary diagnosis of congestive heart failure, coronary revascularization, resuscitated cardiac arrest), (6) individual cerebrovascular events (fatal and non-fatal stroke, fatal and non-fatal ischemic stroke, fatal and non-fatal hemorrhagic stroke, and transient ischemic attack requiring hospital admission), and (7) other events (operation or rupture of aortic aneurysms, aortic dissection, revascularization for peripheral artery disease, carotid endarterectomy or stenting, venous thromboembolism, new onset of malignancy, surgery for aortic stenosis).

### Baseline Data:

The study population was comprised of patients from 768 institutions. The first patient was recruited in January 2010. Randomizations occurred from April 2010 to July 2013. Of the 14,774 patients who entered the open-label run-in period, 13,054 were randomized to pitavastatin 1 mg or 4 mg. The randomized patients who withdrew agreement in an early stage and violated entry criteria were excluded. The baseline characteristics of these 12,413 patients are shown in Table 1, where BMI indicates body mass index; UA, unstable angina; PCI, percutaneous coronary intervention; CABG, coronary artery bypass grafting; CAD, coronary artery disease; LDL-C, low-density lipoprotein cholesterol; HDL-C, high-density lipoprotein cholesterol; Apo, apolipoprotein; hsCRP, high sensitivity C-reactive protein; IQR, interquartile range; and HbA1c, hemoglobin A1c.

TABLE 1

| | n = 12,413 |
|---|---|
| Variable | |
| Age (years) | 68.1 ± 8.3 |
| Men (%) | 82.6 |
| Height (cm) | 162.4 ± 8.1 |
| Body weight (kg) | 65.2 ± 11.3 |
| BMI (kg/m²) | 24.6 ± 3.4 |
| Cardiovascular history | |
| Hospitalization for UA (%) | 25.5 |
| Myocardial infarction (%) | 51.4 |
| Revascularization | |
| PCI (%) | 83.5 |
| CABG (%) | 12.7 |
| Cerebrovascular disease (%) | 8.1 |
| Peripheral arterial disease (%) | 7.0 |
| Diabetes mellitus (%) | 40.1 |
| Current smoker (%) | 16.4 |
| Former smoker (%) | 49.4 |
| Hypertension (%) | 75.7 |
| Family history of CAD (%) | 16.5 |
| History of congestive heart failure (%) | 5.2 |
| Atrial fibrillation/flutter (%) | 6.2 |
| History of malignancy (%) | 5.3 |
| Baseline statin use (%) | 90.9 |
| Total cholesterol (mg/dL) | 166.8 ± 24.3 |
| LDL-C (mg/dL) | 87.9 ± 18.9 |
| HDL-C (mg/dL) | 50.7 ± 12.6 |
| Triglycerides (mg/dL), median (IQR) | 124 (89-174) |
| hsCRP (mg/L), median (IQR) | 0.52 (0.25-1.19) |
| Glucose (mg/dL) | 124.1 ± 40.3 |
| HbA1c (%) | 5.86 ± 0.85 |
| Systolic blood pressure (mmHg) | 127.6 ± 16.2 |
| Diastolic blood pressure (mmHg) | 73.0 ± 10.8 |
| Heart rate | 69.5 ± 11.6 |

### Results:

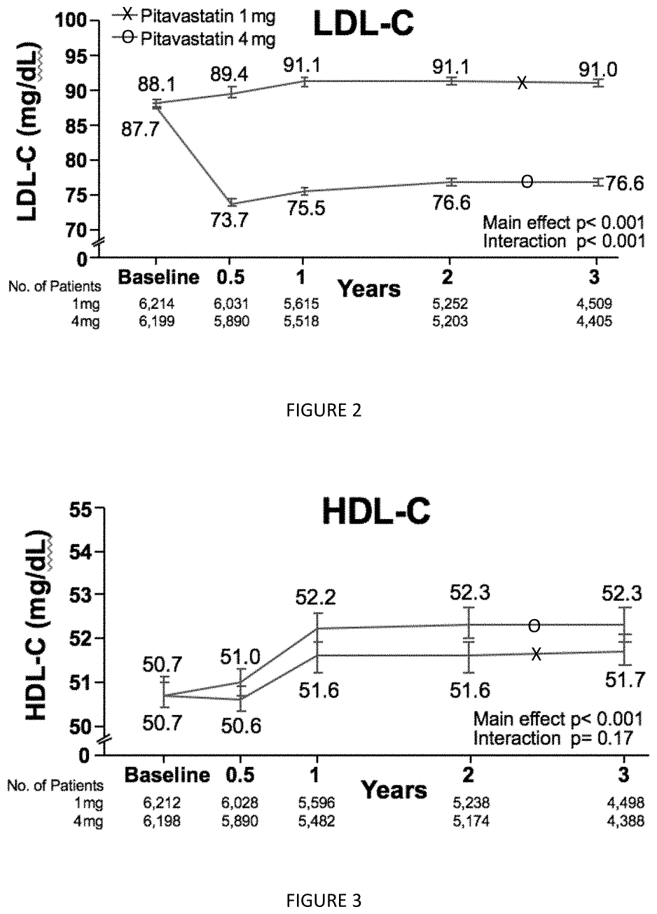

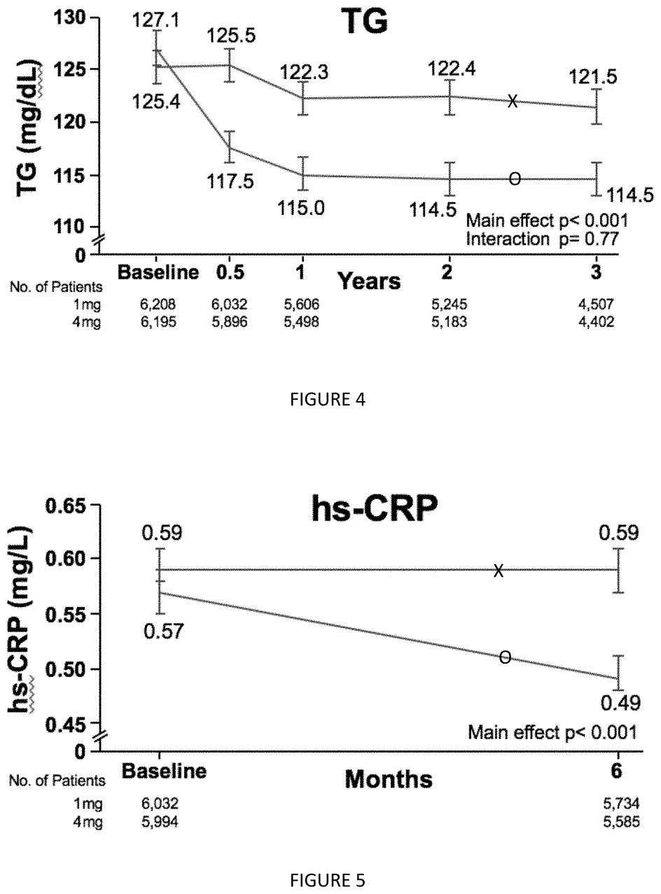

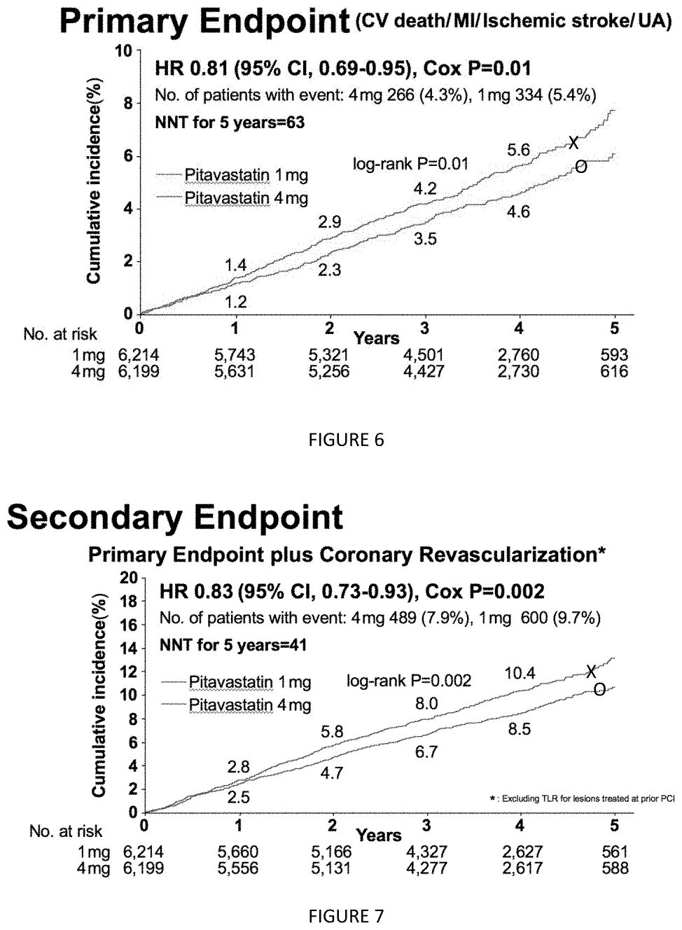

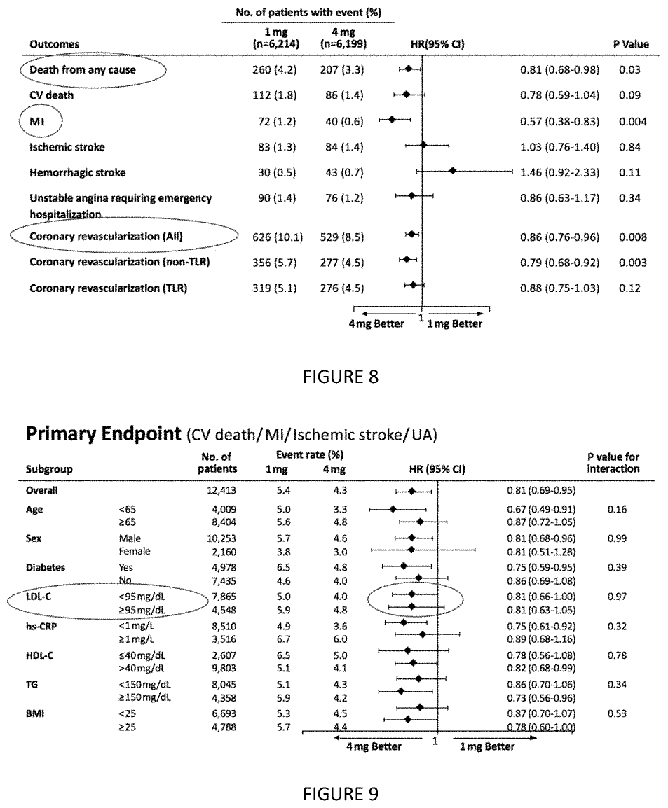

The results of the study are reported in FIGS. 2-9, where one can observe:
a statistically significant reduction in LDL-C when patients were converted from a 1 mg dose of pitavastatin to a 4 mg dose (FIG. 2);
a numerically superior increase in HDL-C when patients were converted from a 1 mg dose of pitavastatin to a 4 mg dose (FIG. 3);
a statistically significant reduction in TG when patients were converted from a 1 mg dose of pitavastatin to a 4 mg dose (FIG. 4);
a statistically significant reduction in hs-CGP when patients were converted from a 1 mg dose of pitavastatin to a 4 mg dose (FIG. 5);
a statistically significant reduction in the primary clinical endpoint (composite of CV death, MI, ischemic stroke and UA) when patients were converted from a 1 mg dose of pitavastatin to a 4 mg dose (FIG. 6);
a statistically significant reduction in a secondary clinical endpoint (primary endpoint+coronary revascularization) when patients were converted from a 1 mg dose of pitavastatin to a 4 mg dose (FIG. 7);
incidence rates of individual CV events in the 1 mg and 4 mg pitavastatin groups, with particular emphasis on the positive death from any cause, myocardial infarction, and coronary revascularization results (FIG. 8); and
incidence rates for the primary endpoint categorized based on various patient characteristics, with particular emphasis on the LDL-C subgroups, showing that LDL-C status (whether ≥95 mg/dL or <95 mg/dL) did not affect the results (FIG. 9).

### REFERENCES CITED

1. Mabuchi H, Kita T, Matsuzaki M, et al. Large scale cohort study of the relationship between serum cholesterol concentration and coronary events with low-dose simvastatin therapy in Japanese patients with hypercholesterolemia and coronary heart disease: secondary prevention cohort study of the Japan Lipid Intervention Trial (J-LIT). Circ J 2002; 66: 1096-100.
2. Japanese Coronary Artery Disease (JCAD) Study Investigators. Current status of the background of patients with coronary artery disease in Japan. Circ J 2006; 70: 1256-62.
3. Yokoyama M, Origasa H, Matsuzaki M, et al. Effects of eicosapentaenoic acid on major coronary events in hypercholesterolaemic patients (JELIS): a randomised open-label, blinded endpoint analysis. Lancet 2007; 369: 1090-8.
4. Scandinavian Simvastatin Survival Study Group. Randomised trial of cholesterol lowering in 4444 patients with coronary heart disease: the Scandinavian Simvastatin Survival Study (4S). Lancet 1994; 344: 1383-9.
5. Sacks F M, Pfeffer M A, Moye L A, et al. The effect of pravastatin on coronary events after myocardial infarction in patients with average cholesterol levels. Cholesterol and Recurrent Events Trial investigators. N Engl J Med 1996; 335: 1001-9.
6. West of Scotland Coronary Prevention Study Group. Influence of pravastatin and plasma lipids on clinical events in the West of Scotland Coronary Prevention Study (WOSCOPS). Circulation 1998; 97: 1440-5.
7. The Long-Term Intervention with Pravastatin in Ischaemic Disease (LIPID) Study Group. Prevention of cardiovascular events and death with pravastatin in patients with coronary heart disease and a broad range of initial cholesterol levels. N Engl J Med 1998; 339: 1349-57.
8. Heart Protection Study (HPS) Collaborative Group. MRC/BHF Heart Protection Study of cholesterol lowering with simvastatin in 20,536 high-risk individuals: a randomised placebo-controlled trial. Lancet 2002; 360: 7-22.
9. Sever P S, Dahlof B, Poulter N R, et al. Prevention of coronary and stroke events with atorvastatin in hypertensive patients who have average or lower-than-average cholesterol concentrations, in the Anglo-Scandinavian Cardiac Outcomes Trial-Lipid Lowering Arm (ASCOT-LLA): a multicentre randomised controlled trial. Lancet 2003; 361: 1149-58.
10. Colhoun H M, Betteridge D J, Durrington P N, et al. Primary prevention of cardiovascular disease with atorvastatin in type 2 diabetes in the Collaborative Atorvastatin Diabetes Study (CARDS): multicentre randomised placebo-controlled trial. Lancet 2004; 364: 685-96.
11. Nakamura H, Arakawa K, Itakura H, et al. Primary prevention of cardiovascular disease with pravastatin in Japan (MEGA Study): a prospective randomised controlled trial. Lancet 2006; 368: 1155-63.
12. Sakamoto T, Kojima S, Ogawa H, et al. Effects of early statin treatment on symptomatic heart failure and ischemic events after acute myocardial infarction in Japanese. Am J Cardiol 2006; 97: 1165-71.
13. Teramoto T, Sasaki J, Ishibashi S, et al. Executive summary of Japan Atherosclerosis Society (JAS) guideline for diagnosis and prevention of atherosclerosis cardiovascular diseases in Japan2012 version. J Atheroscler Thromb 2012; 20: 517-23.
14. Piepoli M F, Hoes A W, Agewall S, et al. 2016 European Guidelines on cardiovascular disease prevention in clinical practice: The Sixth Joint Task Force of the European Society of Cardiology and Other Societies on Cardiovascular Disease Prevention in Clinical Practice (constituted by representatives of 10 societies and by invited experts): Developed with the special contribution of the European Association for Cardiovascular Prevention & Rehabilitation (EACPR). Eur Heart J 2016; 37: 2315-81.
15. Grundy S M, Cleeman J I, Merz C N, et al. Implications of Recent Clinical Trials for the National Cholesterol Education Program Adult Treatment Panel III Guidelines. Circulation 2004; 110: 227-39.
16. de Lemos J A, Blazing M A, Wiviott S D, et al. Early intensive vs a delayed conservative simvastatin strategy in patients with acute coronary syndromes: phase Z of the A to Z trial. JAMA 2004; 292: 1307-16.
17. LaRosa J C, Grundy S M, Waters D D, et al. Intensive lipid lowering with atorvastatin in patients with stable coronary disease. N Engl J Med 2005; 352: 1425-35.
18. Pedersen T R, Faergeman O, Kastelein J J, et al. High-dose atorvastatin vs usual-dose simvastatin for secondary prevention after myocardial infarction: the IDEAL study: a randomized controlled trial. JAMA 2005; 294: 2437-45.
19. Cannon C P, Braunwald E, McCabe C H, et al. Intensive versus moderate lipid lowering with statins after acute coronary syndromes. N Engl J Med 2004; 350: 1495-504.
20. Cannon C P, Steinberg B A, Murphy S A, Mega J L, Braunwald E. Meta-analysis of cardiovascular outcomes trials comparing intensive versus moderate statin therapy. J Am Coll Cardiol 2006; 48: 438-45.
21. Josan K, Majumdar S R, McAlister F A. The efficacy and safety of intensive statin therapy: a meta-analysis of randomized trials. CMAJ 2008; 178: 576-84.
22. Stone N J, Robinson J, Lichtenstein A H, et al. 2013 ACC/AHA guideline on the treatment of blood cholesterol to reduce atherosclerotic cardiovascular risk in adults: a report of the American College of Cardiology/American Heart Association Task Force on Practice Guidelines. J Am Coll Cardiol 2014; 63: 2889-934.
23. Kaneko H, Yajima J, Oikawa Y, et al. Recent characteristics and outcomes of Japanese stable angina pectoris after percutaneous coronary intervention. An observational cohort study using the Shinken Database. Int Heart J 2013; 54: 335-40.
24. Natsuaki M, Furukawa Y, Morimoto T, et al. Intensity of statin therapy, achieved low-density lipoprotein cholesterol levels and cardiovascular outcomes in Japanese patients after coronary revascularization. Perspectives from the CREDO-Kyoto registry cohort-2. Circ J 2012; 76: 1369-79.
25. Okazaki S, Yokoyama T, Miyauchi K, et al. Early statin treatment in patients with acute coronary syndrome: demonstration of the beneficial effect on atherosclerotic lesions by serial volumetric intravascular ultrasound analysis during half a year after coronary event: the ESTABLISH Study. Circulation 2004; 110: 1061-8.
26. Hiro T, Kimura T, Morimoto T, et al. Effect of intensive statin therapy on regression of coronary atherosclerosis in patients with acute coronary syndrome. J Am Coil Cardiol 2009; 54: 293-302.
27. Takayama T, Hiro T, Yamagishi M, et al. Effect of rosuvastatin on coronary atheroma in stable coronary artery disease: multicenter coronary atherosclerosis study measuring effects of rosuvastatin using intravascular ultrasound in Japanese subjects (COSMOS). Circ J 2009; 73: 2110-7.
28. Dohi T, Miyauchi K, Okazaki S, et al. Plaque regression determined by intravascular ultrasound predicts long-term outcomes of patients with acute coronary syndrome. J Atheroscler Thromb 2011; 18: 231-9.
29. Ose L, Budinski D, Hounslow N, Arneson V. Long-term treatment with pitavastatin is effective and well tolerated by patients with primary hypercholesterolemia or combined dyslipidemia. Atherosclerosis 2010; 210: 202-8.
30. Austen W G, Edwards J E, Frye R L, et al. A reporting system on patients evaluated for coronary artery disease. Report of the Ad Hoc Committee for Grading of Coronary Artery Disease, Council on Cardiovascular Surgery, American Heart Association. Circulation 1975; 51 (4 Suppl): 5-40.

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1) A method of preventing an adverse cardiovascular event in a subject on a dosage of 1 mg/day pitavastatin or a pharmaceutically acceptable salt thereof, wherein the subject has an LDL-C concentration less than 120 mg/dL, comprising increasing the dosage to 4 mg/day for a therapeutically effective period of time.

2) The method of claim 1, wherein the subject has coronary artery disease.

3) A method of preventing an adverse cardiovascular event in a subject with stable coronary artery disease, comprising administering to the subject 4 mg/day of pitavastatin or a pharmaceutically acceptable salt thereof for a therapeutically effective period of time.

4) The method of claim 3, wherein prior to administering the 4 mg/day the subject is on a dosage of 1 mg/day pitavastatin or a pharmaceutically acceptable salt thereof and has an LDL-C concentration less than 120 mg/dL.

5) A method of preventing an adverse cardiovascular event in a subject on a dosage of 1 mg/day pitavastatin or a pharmaceutically acceptable salt thereof, wherein the subject has stable coronary artery disease and an LDL-C concentration less than 120 mg/dL, comprising increasing the dosage to 4 mg/day for a therapeutically effective period of time.

6) (canceled)

7) (canceled)

8) The method of claim 1, wherein the subject has stable coronary artery disease characterized by:
a. previous ACS, such as acute myocardial infarction or unstable angina;
b. a previous coronary revascularization procedure (PCI or coronary artery bypass grafting (“CABG”)); or
c. atherosclerotic CAD detected by coronary artery angiography with at least 75% stenosis in major epicardial coronary arteries.

9) (canceled)

10) (canceled)

11) (canceled)

12) (canceled)

13) (canceled)

14) (canceled)

15) (canceled)

16) (canceled)

17) (canceled)

18) (canceled)

19) (canceled)

20) (canceled)

21) (canceled)

22) (canceled)

23) The method of claim 1, wherein the subject has type II diabetes.

24) (canceled)

25) The method of claim 1, wherein the subject has an LDL-C concentration less than 95 mg/dL.

26) (canceled)

27) (canceled)

28) (canceled)

29) The method of claim 1, wherein the subject has an HDL-C concentration less than or equal to 40 mg/dL.

30) (canceled)

31) The method of claim 1, wherein the subject has an TG concentration less than 150 mg/dL.

32) (canceled)

33) (canceled)

34) (canceled)

35) The method of claim 1, wherein the subject has a body mass index less than 28 kg/m².

36) (canceled)

37) (canceled)

38) (canceled)

39) (canceled)

40) The method of claim 1, wherein the subject does not have a history of acute coronary syndrome (ACS).

41) (canceled)

42) (canceled)

43) (canceled)

44) (canceled)

45) (canceled)

46) (canceled)

47) The method of claim 1, wherein the subject has chronic kidney disease (CKD) (eGFR<60 mL/min/1.73 m²).

48) (canceled)

49) The method of claim 1, wherein the subject is on aspirin therapy.

50) (canceled)

51) The method of claim 1, wherein the subject is on dual antiplatelet therapy (DAPT).

52) (canceled)

53) The method of claim 1, further comprising decreasing LDL-C levels in the subject.

54) The method of claim 1, further comprising increasing HDL-C levels in the subject.

55) The method of claim 1, further comprising decreasing TG levels in the subject.

56) The method of claim 1, further comprising decreasing hs-CRP levels in the subject.
